(19) United States
(12) Patent Application Publication
(10) Pub. No.: US 2004/0002500 A1
(43) Pub. Date: Jan. 1, 2004

(54) Methods for treating attention deficit disorder

(75) Inventors: Stephen J. Kramer, Houston, TX (US); Louis F. Fabre, Houston, TX (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Fabre Kramer Pharmaceutical, Inc., Houston, TX

(21) Appl. No.: 10/183,527

(22) Filed: Jun. 28, 2002

(51) Int. Cl. A61K 31/506; A61K 31/4747

(52) U.S. Cl. 514/252.15; 514/278

(57) ABSTRACT

The present invention relates to a method for treatment of attention deficit disorder by administering certain 5-HT_{1A} receptor agonists

### BACKGROUND OF THE INVENTION

1. Field of the Invention

The present invention relates to a method for alleviation, prevention, and treatment of attention deficit disorder by administering certain 5-HT_{1A} receptor agonists.

2. Discussion of the Background

Attention deficit disorder (ADD) is a learning disorder that relates to developmentally inappropriate inattention and impulsivity. ADD may also be referred to as disruptive behavior disorder or minimal brain dysfunction. ADD may be present with or without hyperactivity. A common disorder, ADD accounts for more child mental health referrals than any other single disorder. ADD is estimated to affect 3 to 5% of school-aged children, and is much more frequent in males than in females, with a male-to-female ratios ranging from 4:1 to 9:1. On the average, at least one child in every classroom in the United States needs help for the disorder. ADD often continues into adolescence and adulthood, and can cause a lifetime of frustrated dreams and emotional pain. In addition, ADD may affect the behavior of children at any cognitive level.

ADD is a diagnosis applied to children and adults who consistently display certain characteristic behaviors over a period of time. The most common behaviors fall into two categories: inattention and impulsivity. Attention deficit disorder with hyperactivity is diagnosed when the signs of overactivity are obvious. Inappropriate inattention causes increased rates of activity and impersistence or reluctance to participate or respond. A subject suffering from ADD exhibits a consistent pattern of inattention and/or hyperactivity-impulsivity that is more frequent and severe than is typically observed in individuals at a comparable level of development. Such subjects must suffer clear evidence of interference with developmentally appropriate social, academic, or occupational functioning.

Although subjects with ADD and without hyperactivity may not manifest high activity levels, most exhibit restlessness or jitteriness, short attention span, and poor impulse control. These are qualitatively different from those seen in conduct and anxiety disorders. Inattention is described as a failure to finish tasks started, easy distractibility, seeming lack of attention, and difficulty concentrating on tasks requiring sustained attention. Impulsivity is described as acting before thinking, difficulty taking turns, problems organizing work, and constant shifting from one activity to another. Impulsive responses are especially likely when involved with uncertainty and the need to attend carefully. Hyperactivity is featured as difficulty staying seated and sitting still, and running or climbing excessively. A more complete description of the symptoms and diagnostic criteria of attention deficit disorder with or without hyperactivity are provided by DSM-IV (Diagnostic and Statistical Manual of Mental Disorders, 1994; 78-85), which is incorporated herein by reference.

No single treatment has been completely effective for attention deficit disorder. Psychostimulant medications combined with behavioral and cognitive therapies (e.g., selfrecording, self-monitoring, modeling, and role-playing) have the greatest controlling influence on symptom expression. Used alone, medication has been effective predominantly with less aggressive ADD children coming from stable home environments. Elimination diets, megavitamin treatments, psychotherapy, and biochemical interventions (e.g., the administration of neurochemicals) have had only minor, unsustained effects.

For decades, medications have been used to treat the symptoms of ADD. The three most common medications in both adults and children are the stimulants; methylphenidate (RITALIN™), dextroamphetamine (DEXEDRINE™ or DEXTROSTAT™), and pemoline (CYLERT™). For many people, these medicines dramatically reduce their hyperactivity and improve their ability to focus, work, and learn. The medications may also improve physical coordination, such as handwriting and ability in sports. Recent research by National Institute of Mental Health suggests that these medicines may also help children with an accompanying conduct disorder to control their impulsive, destructive behaviors. Nine out of 10 children improve on one of these three stimulant drugs.

Different doctors use the medications in slightly different ways. CYLERT™ is available in one form, which naturally lasts 5 to 10 hours. RITALIN™ and DEXEDRINE™ come in short-term tablets that last about 3 hours, as well as longer-term preparations that last through the school day.

Stimulant drugs, when used with medical supervision, are usually considered safe. However, a common problem with stimulant drugs is that they can be addictive to teenagers and adults if misused. While on these medications, some children may lose weight, have less appetite, and temporarily grow more slowly. Others may have problems falling asleep. Some doctors believe that stimulants may also make the symptoms of Tourette's syndrome worse.

The most commonly prescribed ADD medication is RITALIN™, which is generally more effective than tricyclic antidepressants (e.g., IMIPRAMINE™), caffeine, and other psychostimulants (e.g., PEMOLINE™ and DEANOL™) and has fewer side effects than dextroamphetamine. Common side effects of RITALIN™ are sleep disturbances (e.g., insomnia), depression or sadness, headache, stomachache, suppression of appetite, elevated blood pressure, and, with large continuous doses, a reduction of growth. Long-term benefits of medication with RITALIN™, however, have not been demonstrated conclusively. Some research indicates that use of medication permits participation in activities previously inaccessible because of poor attention and impulsivity.

The frequency of side effects, potential addictiveness, and limited success of stimulant drugs has led to a search for alternate means of treating or preventing attention deficit disorders.

One such strategy has been to target the dopaminergic system. Deregulation of the dopaminergic system has been linked with Parkinson's disease, Tourette's syndrome, schizophrenia, attention deficit hyperactive disorder (ADHD) and generation of pituitary tumours (Vallone et al, Neurosci Biobehav Rev January 2000;24: 125-32). The azapirone, buspirone, has been speculated as having a potential therapeutic role in treatment of ADHD (Balon, J. Clin. Psychopharma. 1990; 10: 77, and Malhotra et al, J. Am. Acad. Child Adolesc. Psychiatry 1998; 57: 364-371).

Buspirone exhibits an affinity for a series of receptors including serotonin receptors, dopamine receptors, and α-adrenergic receptors. The effect of buspirone on the dopaminergic system occurs by enhancing dopamine synthesis and release (Tunnicliff et al, Neuropharmacology 1992; 31: 991-5). Buspirone blocks the presynaptic dopaminergic receptors rather than the postsynaptic dopaminergic receptors, thereby increasing the firing of the midbrain neurons and blocking the inhibiting effects of γ-aminobutyric acid on dopaminergic neurons in the zona compacta of the substantia nigra (Eison and Temple, Am. J. Med. 1986; 80(3B suppl): 1-9). Although the serotonergic activity may be related to improved behavior and impulsivity, according to Balon and Malhotra et al, the dopaminergic activity of buspirone leads to improved attention span and decreased hyperactivity associated with ADHD. However, more recent reports on treatment strategies of attention deficit disorder have suggested that buspirone may have a potentially deleterious effect on patients having ADHD (Popper, Child Adolesc Psychiatr Clin N Am 2000; 9: 605-46).

Accordingly, there remains a critical need for novel treatment strategies of patients suffering from ADD, with or without hyperactivity. Moreover, there remains a critical need for treatment strategies, which are safe and effective with a reduction in or elimination of any side effects associated with existing treatment strategies.

### SUMMARY OF THE INVENTION

Accordingly, it is an object of the present invention to provide methods for treatment of attention deficit disorder, with or without hyperactivity, or symptoms thereof. This object can be achieved by administering to a patient in need thereof with a 5-HT_{1A} receptor agonist.

The present invention is based, in part, on the discovery that a patient suffering from attention deficit disorder can be treated by an azapirone 5-HT_{1A} receptor agonist, which lacks dopamine receptor activity. Accordingly, it is an object of the present invention to treat a patient having attention deficit disorder, with or without hyperactivity, or symptoms thereof, with an azapirone 5-HT_{1A} receptor agonist, which lacks dopamine receptor activity. Examples of suggested azapirone 5-HT_{1A} receptor agonists, which lack dopamine receptor activity, include gepirone, ipsapirone, and tandospirone.

It is another object of the present invention to treat a patient having attention deficit disorder, with or without hyperactivity, or symptoms thereof, with an adamantyl aryl- or heteroaryl piperazinyl carboxamide 5-HT_{1A} receptor agonist. One example of this 5-HT_{1A} receptor agonist is adatanserin.

It is another object of the present invention to treat a patient having attention deficit disorder, with or without hyperactivity, or symptoms thereof, with a hetrobicyclic-arylpiperazine 5-HT_{1A} receptor agonist. One example of this 5-HT_{1A} receptor agonist is flesinoxan.

In one embodiment, the 5-HT_{1A} receptor agonist is administered in conjunction with an agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

In another embodiment, the 5-HT_{1A} receptor agonist is administered with the administration of methylphenidate (RITALIN™).

In another embodiment, the 5-HT_{1A} receptor agonist is administered with a pharmaceutically acceptable carrier.

In another embodiment, the 5-HT_{1A} receptor agonist is administered orally, rectally, nasally, parenterally, intracisternally, intravaginally, intraperitoneally, sublingually, topically, or bucally.

In another embodiment, the therapeutically effective amount of the 5-HT_{1A} receptor agonist is similar to the anxiolytic dose of the medication, e.g.: 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day) in single or multiple doses.

In another embodiment, the total daily dose of including ipsapirone and tandospirone administered to a patient in need thereof, in single or in divided doses, can be in amounts of 0.25-3.0 mg/kg of body weight/day.

In another embodiment, the patient in need thereof also suffers from one or more of anxiety, depression, obesity, drug abuse/addiction, alcohol abuse, sleep disorders, TIC disorder, and behavioral/cognitive symptoms of Alzheimer's disease.

Another object of the present invention is to provide a treatment regimen of concurrently administering to a patient in need thereof mixtures of two or more of the compounds of the present invention.

Yet another object of the present invention is to provide a treatment regimen of administering to a patient in need thereof a single or divided dose of a first compound followed by, on the same day or a subsequent day, a single or divided dose of one or more additional compounds.

The above object highlights certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1: The 5-HT (serotonin) neuron in the hyperactive state: namely, high levels of 5-HT, overactive 5-HT neuronal firing and transmission, and down-regulated somatodendritic presynaptic 5-HT_{1A} autoreceptors.

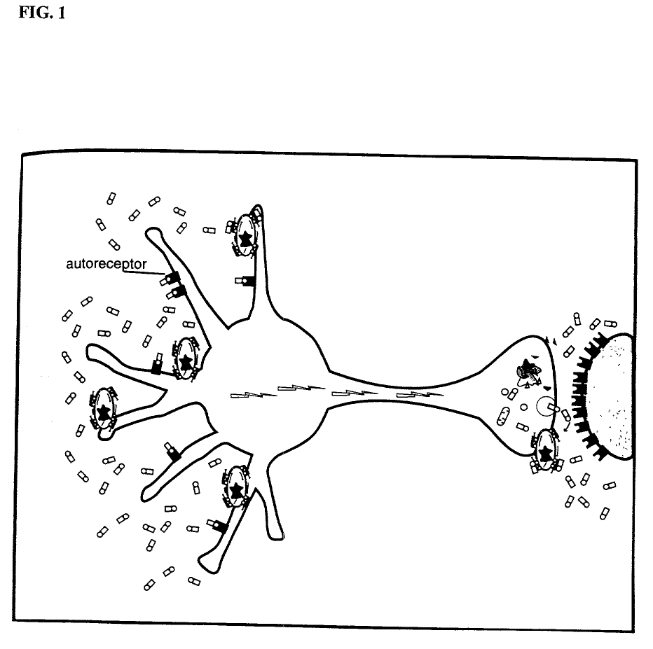

FIG. 2: The drugs (gepirone, ipsapirone, tandospirone, flesinoxan, and adatanserin) are presynaptic agonists and post-synaptic partial agonists. A presynaptic agonist shuts off the neuron (less serotonin post-synaptically). Post-synaptic partial agonism results in less robust stimulation of the post-synaptic neuron than serotonin itself (resulting in less firing). The net result is less post-synaptic neuron firing.

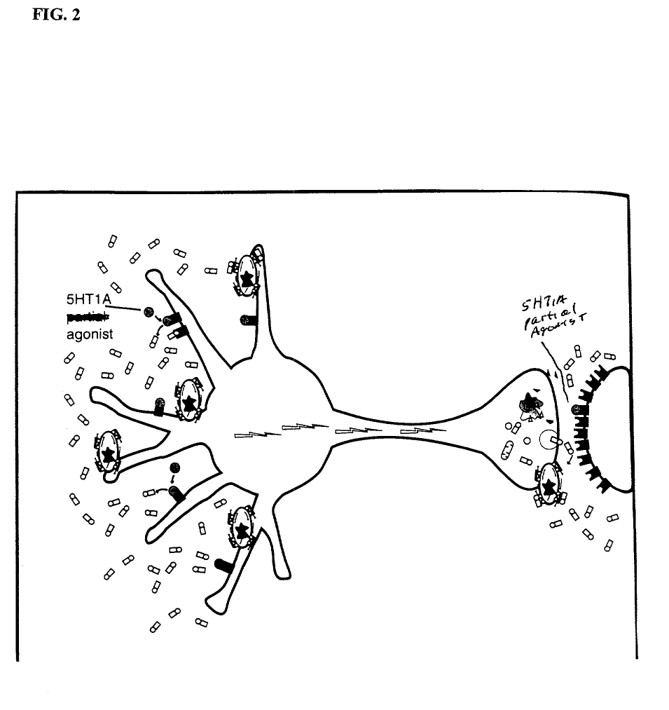

### DETAILED DESCRIPTION OF THE INVENTION

Unless specifically defined, all technical and scientific terms used herein have the same meaning as commonly understood by a skilled artisan in organic chemistry, biochemistry, psychology, psychiatry, medicine, neurochemistry, and neurology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

As used herein, the general term “attention deficit disorder” includes attention deficit disorder and disruptive behavior disorder each of which may be present with or without hyperactivity.

The term “5-HT_{1A} receptor agonist” means partial agonist or full agonist with respect to either presynaptic receptors or postsynaptic receptors, or both, unless otherwise specified.

In psychopharmacology, the serotonin (5-hydroxytryptamine [5-HT]) type-1A (5-HT_{1A}) receptor has acquired considerable attention as a therapeutic target. Agonism of the 5-HT_{1A} receptor has been correlated with treatment of anxiety, depression, obesity, drug abuse/addiction, alcohol abuse, sleep disorders and behavioral and cognitive symptoms of Alzheimer's disease (U.S. Pat. No. 4,423,049, US patent 4,771,053, US patent 5,106,849, and Eison, Journal of Clinical Psychopharma, 1990; 10: S2-S5). Accordingly, in an embodiment of the present invention is to treat a patient suffering from attention deficit disorder and at least one of the above-stated disorders by administering a therapeutically effective amount of the compounds and/or preparations of the present invention.

The use, preparation, and characterization of therapeutic azapirone compounds have been disclosed in numerous documents (see Cadieux, Amer. Family Physician 1996 53: 2349-2353; Temple, U.S. Pat. No. 4,423,049; Gawin, U.S. Pat. No. 5,185,329; Madding, U.S. Pat. No. 5,521,313). This class of compounds attributes its activity to partial agonism of the 5-HT_{1A} receptor.

Clinical studies of known 5-HT_{1A} agonists and partial agonists, for example buspirone, ipsapirone, and gepirone, have shown that these compounds are useful in the treatment of anxiety disorders, such as generalized anxiety disorder (GAD), panic disorder, and obsessive compulsive disorder (Glitz. D. A., Pohl, R., Drugs 1991, 41:1 1; Cadieux, Amer. Family Physician 1996 53: 2349-2353). Clinical and preclinical evidence supports 5-HT_{1A} partial agonists for use in treating depression as well as impulse control disorders and alcohol abuse (van Hest, Psychopharm., 107: 474 (1992); Schipper et al, Human Psychopharm., 6: S53 (1991); Cervo et al, Eur. J. Pharm., 158: 53 (1988); Glitz, D. A., Pohl, R., Drugs, 41: 11(1991)). Studies show that 5-HT_{1A} agonists and partial agonists inhibit isolation-induced aggression in male mammals, indicating that they can be used to treat aggression (Sanchez et al. Psychopharmacology, 1993, 110:53-59). Other studies indicate that 5-HT_{1A} receptors are important in the serotonergic modulation of haloperidol-induced catalepsy (Hicks, Life Science 1990, 47:1609) suggesting that 5-HT_{1A} agonists can be used to treat the deleterious side effects of conventional antipsychotic agents, such as haloperidol. Recent reports show that this is the case for side effects like tardive dyskinesias.

The present invention is based, in part, on the Inventor's surprising discovery that certain 5-HT_{1A} receptor agonists can be employed to treat attention deficit disorder, with or without hyperactivity, or symptoms thereof. Specifically, this object can be achieved by administering to a patient in need thereof a therapeutically effective amount of a 5-HT_{1A} receptor agonist selected from an azapirone 5-HT_{1A} receptor agonist, which lacks dopamine receptor activity, a hetrobicyclic-aryl-piperazine 5-HT_{1A} receptor agonist, or an adamantyl aryl- or heteroaryl piperazinyl carboxamide 5-HT_{1A} receptor agonist.

In one embodiment, the azapirone 5-HT_{1A} receptor agonist, which lacks dopamine receptor activity, is gepirone. Gepirone (also known as 4,4-dimethyl-1-[4-[4-(2-pyrimidinyl)1-piperazinyl]-butyl]-2,6-piperadinedione hydrochloride) can be obtained by the process according to Temple (U.S. Pat. No. 4,423,049, which is incorporated herein in its entirety by reference) and has the following structure:

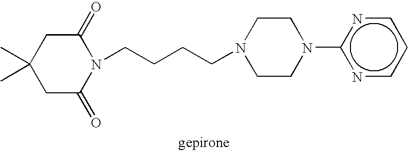

Briefly, a solution of 3,3-dimethylglutaric anhydride and 1-(4-aminobutyl)-4-(2-pyrimidinyl)piperazine in xylene is refluxed, collecting water of reaction by means of a Dean Stark trap. The reaction mixture is then filtered while warm (approximately 80° C.) and the filtrate concentrated in vacuo to give a residue. The residue is then distilled and the distillation product is either crystallized from acetonitrile to give a solid base, or is treated with ethanolic HCl to give the hydrochloride salt.

In other embodiments, ipsapirone (2-(4-(4-(2-pyrimidinyl)-1-piperazinyl)-butyl)1,2-benzoisothiazol-3 (2H)-one 1,1-dioxide hydrochloride) and tandospirone (N-[4-[4-(2-pyrimidinyl)-1-piperazinyl]butyl]-2,3-norbornanedicarboximide). Ipsapirone and methods of making ipsapirone are disclosed in U.S. Pat. No. 4,988,700 and Traber et al (Trends Pharmacol Sci 1987; 8: 432-7), both of which are incorporated herein by reference; tandospirone and methods of making tandospirone are disclosed in U.S. Pat. Nos. 4,507,303; 4,543,355; 4,598,078 and 5,011,841, all of which are incorporated herein by reference.

Methods of assessing the receptor (5-HT_{1A} and dopamine) agonistic activity of the compounds of the present invention are well known to those of skill in the art. These methods can be found in Iser-Strenegr, et al (Brain Res November 1986;395(t):57-65), Millan, et al (J Pharmacol Exp Ther March 1993;264(3):1364-76), and Perrone R, et al (J Med Chem Mar. 17, 1995;38(6):942-9), all of which are incorporated herein by reference.

Flesinoxan ((+)-N-[2-[4-(2,3-dihydro-2-hydroxymethyl-1,4-benzodioxin-5-yl)-1piperaxinyl]ethyl]-4-fluorobenzamide hydrochloride) is a high affinity and high selectivity 5-HT_{1A} receptor agonist, which has been shown to have both anxiolytic and antidepressant activity (Hadrava et al, Neuropharmacology, 1995; 34(10): 1311-1326). Accordingly, in another embodiment of the present invention the 5-HT_{1A} receptor agonist that can be employed to treat attention deficit disorder, with or without hyperactivity, or symptoms thereof, is the hetrobicyclic-aryl-piperazine 5-HT_{1A} receptor agonists. Examples of other suitable compounds of this class, as well as methods of making these compounds, are disclosed in Hartog et al (U.S. Pat. No. 4,833,142, which is incorporated herein by reference).

In another embodiment of the present invention, a patient having attention deficit disorder, with or without hyperactivity, or symptoms thereof, can be administered an adamantyl aryl- or heteroaryl piperazinyl carboxamide 5-HT_{1A} receptor agonist to treat the disorder. As a specific example of this class of compounds, adatanserin (N-[2-[4-(2-pyrimidinyl)-1-piperazinyl]ethyl]tricyclo[3.3.1.1^{3,7} ]decane-1-carboxamide) is mentioned. Adamantyl aryl- or heteroaryl piperazinyl carboxamide 5-HT_{1A} receptor agonist compounds envisioned as suitable for administration in accordance with this invention can be synthesized as described by Abou-Gharbia, et al (U.S. Pat. Nos. 5,106,849; 5,010,078; 5,380,725; 5,482,940; 5,278,160; 5,254,552; and J Med Chem Dec. 16, 1999;42(25):5077-94, all of which are incorporated herein by reference).

A drug that treats attention deficit disorder is assessed by measuring the child's behavior before and after treatment with a drug of the present invention. Measurements of the child's behavior include clinical measures and rating scales. Two clinical measures are the simulated classroom (Gadow et al, Stony Brook, N.Y.: Checkmate Plus, 1996) and the continuous performance test (Roberts et al, J Pediatr Psychol 1984;9:177-191, Halperin et al, J Am Acad Child Adolesc Psychiatry 1992;31:190-196, and Halperin et al, J Am Acad Child Adolesc Psychiatry 1988;27:326-329).

The simulated classroom requires the child to sit alone at a desk in a small classroom completing work, and not playing with toys on an adjacent table. Clinic sessions are video-recorded through a one-way window to facilitate ease of scoring. The 3 ADHD behaviors measured are Off Task, Fidgeting, and Worksheets (number of items completed correctly). The continuous performance test (CPT) requires a child to press the space bar whenever the letter “A” followed the letter “X” on a computer screen. The CPT generates 3 scores (inattention, impulsivity, and dyscontrol) and takes approximately 12 minutes to complete. Examples of rating scales include the Abbreviated Teacher Questionnaire (ATQ; Conners, Psychopharm Bull 1973;9:24-84 and Epstein et al, J Special Educ 1986;20:219-229), the Iowa-Corners Teachers Rating Scale (Loney et al, Advances in developmental and behavioral pediatrics 1982; vol. 3, Greenwich, Conn.: JAI Press; 113-147), and the Primary Secondary Symptom Checklist (Loney, Poster presented at the annual meeting of the American Psychological Association, Toronto, Ontario, 1984). Normally both teachers and parents scales are rated.

The results of the pre-treatment and post-treatment evaluations can be analyzed by appropriate statistical procedures, such as those contained in Mandel, The Statistical Analysis of Experimental Data, Dover Publications; Toronto, Ontario, 1964.

Based on the Inventor's surprising discovery it can be surmised that psychostimulants, such as methylphenidate (RITALIN™) work because they stimulate inhibitory pathways, whereas the serotonin partial agonists of the present invention work because they inhibit stimulatory pathways.

One skilled in the art is familiar with numerous methods for designing and optimizing formulations and delivery methods to deliver 5-HT_{1A} receptor agonists, in particular gepirone, ipsapirone, tandospirone, flesinoxan, and adatanserin in effective and non-toxic ways. Remington's Pharmaceuticals Sciences, 118th Edition (specifically incorporated herein by reference), can be relied on and used for these purposes, especially Part 8 therein, “Pharmaceutical Preparations and Their Manufacture.” The following compounds, compositions, delivery methods, delivery dosages, and formulations are specifically envisioned as suitable for, but not meant to limit, the present invention.

The pharmaceutical compounds suitable for administration in the present invention may be hydrochloride salts, but the free bases and other pharmaceutically acceptable salts are also suitable. The term “pharmaceutically acceptable salt” is well known in the art, as described in S. M. Berge, et al. (J Pharmaceutical Sciences, 66: 1-19, 1977). Suitable pharmaceutically acceptable salts for administration in the present invention include acid addition salts. The acid addition salt may be formed by mixing a solution of the compound with a solution of a pharmaceutically acceptable non-toxic acid such as hydrochloric acid, hydrobromic acid, fumaric acid, maleic acid, succinic acid, acetic acid, citric acid, tartaric acid, carbonic acid, phosphoric acid, perchloric acid, sulphuric acid, oxalic acid, or malonic acid. Where the compound carries an acidic group, for example a carboxylic acid group, the present invention also contemplates salts thereof, preferably nontoxic pharmaceutically acceptable salts thereof, such as the sodium, potassium and calcium salts thereof.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, furnarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, salts of amine groups. Salts of amine groups may also comprise the quaternary ammonium salts in which the amino nitrogen atom carries an alkyl, alkenyl, alkynyl or aralkyl group, nontoxic ammonium, quaternary ammonium, and amine cations formed using counterions such as halide, hydroxide, carboxylate, sulfate, phosphate, nitrate, loweralkyl sulfonate and aryl sulfonate.

A therapeutically effective amount of the pharmaceutical compounds suitable for administration in the present invention may be administered alone or in combination with one or more pharmaceutically acceptable carriers. As used herein, the term “pharmaceutically acceptable carrier” means a non-toxic, inert solid, semi-solid or liquid filer, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions suitable for administration in the invention can be administered to patients in need thereof orally, rectally, nasally, parenterally (e.g., intramuscular, intraperitoneal, intravenous or subcutaneous injection, or implant), intracisternally, intravaginally, intraperitoneally, sublingually, topically (e.g., as a powder, ointment, or drop), bucally, as an oral spray, or a nasal spray. The pharmaceutical compositions can be formulated in dosage forms appropriate for each route of administration.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art. The inert diluents may include, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The liquid dosage form for oral administration may also contain adjuvants, which include wetting agents, emulsifying and suspending agents, sweetening, flavoring, and perfuming agents. Other dosage forms for oral administration include, for example, aqueous suspensions containing the active compound in an aqueous medium in the presence of a non-toxic suspending agent such as sodium carboxy-methylcellulose, and oily suspensions containing a compound of the present invention in a suitable vegetable oil, for example arachis oil.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This maybe accomplished by the use of a liquid suspension of crystalline or amorphous material with poor water solubility. The rate of absorption of the drug then depends upon its rate of dissolution, which, in turn, may depend upon crystal size and crystalline form. Alternatively, dissolving or suspending the drug in an oil vehicle accomplishes delayed absorption of a parenterally administered drug form. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, prills, powders, and granules. In such solid dosage forms, the active compound is mixed with at least one inert, pharmaceutically acceptable excipient or carrier. In addition, the solid dosage form may contain one or more fillers, extenders, binders, humectants, disintegrating agents, retarding agents, absorption accelerators, wetting agents, absorbents, or lubricants. Examples of suitable fillers or extenders include, starches, lactose, sucrose, glucose, mannitol, and silicic acid, sodium citrate and dicalcium phosphate. Examples of suitable binders include, microcrystalline cellulose, carboxymethylcellulose, alginates, gelatin, polyvinylpyrrolidinone, sucrose, and acacia. Glycerol is an example of a suitable humectant. Examples of suitable disintegrating agents include, agar-agar, calcium carbonate, potato or tapioca starch, maize starch, alginic acid, certain silicates, and sodium carbonate. Paraffin is an example of a suitable solution-retarding agent. As absorption accelerators, any quaternary ammonium compound may be used. Examples of suitable wetting agents include, cetyl alcohol and glycerol monostearate. Examples of suitable absorbents include, kaolin and bentonite clay. Examples of suitable lubricants include, talc, calcium stearate, magnesium stearate, solid polyethylene glycols, sodium lauryl sulfate. In the case of capsules, tablets and pills, the dosage form may also comprise buffering agents.

The tablets may, if desired, be coated using known methods and excipients that may include enteric coating using for example hydroxypropylmethylcellulose phthalate. The tablets may be formulated in a manner known to those skilled in the art so as to give a sustained release of the compounds of the present invention. Such tablets may, if desired, be provided with enteric coatings by known methods, for example by the use of cellulose acetate phthalate. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hardfilled gelatin capsules using such excipients as lactose or milk sugar as well as high molecular weight polyethylene glycols.

If desired, the compounds of the present invention can be incorporated into slow release or targeted delivery systems such as polymer matrices, liposomes and microspheres. They may be sterilized, for example, by filtration through a bacteria-retaining filter, or by incorporating sterilizing agents in the form of sterile solid compositions that can dissolve in sterile water, or some other sterile injectable medium immediately before use.

The active compound may be formulated into granules with or without additional excipients. The granules may be ingested directly by the patient or they may be added to a suitable liquid carrier (for example, water) before ingestion. The granules may contain disintegrates, e.g. an effervescent couple formed from an acid and a carbonate or bicarbonate salt to facilitate dispersion in the liquid medium.

Dosage forms for topical or transdermal administration of a compound of this invention include ointments, pastes, creams, lotions, gels, powders, solutions, sprays, inhalants or patches. Transdermal patches have the added advantage of providing controlled delivery of a compound to the body. The rate can be controlled by either providing a rate controlling membrane or by dispersing the compound in a polymer matrix or gel. The active component is admixed under sterile conditions with a pharmaceutically acceptable carrier and any needed preservatives or buffers as may be required. Dissolving or dispensing the compound in the proper medium can make such dosage forms. Absorption enhancers can also be used to increase the flux of the compound across the skin. Ophthalmic formulation, eardrops, eye ointments, powders and solutions are also contemplated as being within the scope of this invention.

Dosage forms for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as animal and vegetable fats, oils, petrolatum, waxes, paraffins, starch, tragacanth, cellulose derivatives, polyethylene glycols, silicones, bentonites, silicic acid, talc and zinc oxide, or mixtures thereof, together with a potential transdermal accelerant such as dimethyl sulphoxide or propylene glycol. Alternatively the active compounds may be dispersed in a pharmaceutically acceptable paste, cream, gel or ointment base. The amount of active compound contained in a topical formulation should be such that a therapeutically effective amount of the compound is delivered during the period of time for which the topical formulation is intended to be on the skin.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. The therapeutically active compound may be formulated into a composition, which is dispersed as an aerosol into the patient's oral or nasal cavity. Such aerosols may be administered from a pump pack or from a pressurized pack containing a volatile propellant.

The therapeutically active compounds used in the method of the present invention may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as an oily suspension of the compound to be infused for example in the form of a very sparingly water-soluble derivative such as a dodecanoate salt or a lipophilic ester or (b) solid in the form of an implanted support, for example of a synthetic resin or waxy material, for the compound to be infused. The support may be a single body containing the entire compound or a series of several bodies each containing part of the compound to be delivered. The amount of active compound present in an internal source should be such that a therapeutically effective amount of the compound is delivered over a long period of time.

It will be known to those skilled in the art that there are numerous compounds, which may be used for treating attention deficit disorder in a patient. Combinations of these therapeutic agents, some of which have also been mentioned herein, will bring additional, complementary, and often synergistic properties to enhance the desirable properties of these various therapeutic agents. In these combinations, the 5-HT_{1A} agonist and the therapeutic agents may be independently present in dose ranges from one one-hundredth to one times the dose levels which are effective when these compounds are used singly. In such combination therapy, the 5-HT_{1A} agonist may be administered with the other therapeutic agent (e.g., concurrently, concomitantly, sequentially, or in a unitary formulation) such that their therapeutic efficacy overlaps.

The 5-HT_{1A} agonist may be employed in conjunction with an agent selected from the group consisting of stimulants, hypnotics, anxiolytics, antipsychotics, antianxiety agents, minor tranquilizers, benzodiazepines, barbituates, serotonin agonists, selective serotonin reuptake inhibitors, dopamine antagonists, 5-HT_{1A} agonists, 5-HT₂ antagonists, non-steroidal anti-inflammatory drugs, monoamine oxidase inhibitors, muscarinic agonists, norephinephrine uptake inhibitors, essential fatty acids, and neurokinin-1 receptor antagonist.

For example, for treating attention deficit disorder in a patient a 5-HT_{1A} agonist may be given in combination with such compounds as: adinazolam, allobarbital, alonimid, alprazolam, amitriptyline, amobarbital, amoxapine, bentazepam, benzoctamine, brotizolam, bupropion, busprione, butabarbital, butalbital, caffeine, capuride, carbocloral, chloral betaine, chloral hydrate, chlordiazepoxide, clomipramine, cloperidone, clorazepate, clorethate, clozapine, cyprazepam, deanol, desipranune, dexclamol, dextroamphetamine, diazepam, dichloralphenazone, divalproex, diphenhydramine, doxepin, duloxetine, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, methylphenidate (including d-methylphenidate, especially d-methylphenidate hydrochloride), midaflur, midazolam, nefazodone, nisobamate, nitrazepam, nortriptyline, omega-3 fatty acids, oxazepam, paraldehyde, paroxetine, pemoline, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, valproate, venlafaxine, xanomeline, zaleplon, zolazepam, zolpidem, and salts thereof, and combinations thereof, and the like, as well as admixtures and combinations thereof.

As used herein, the term “therapeutically effective amount” refers to that amount of a compound or preparation of the present invention that successfully prevents or reduces the severity of symptoms associated with attention deficit disorder, with or without hyperactivity. This term also embraces the amount of a compound or preparation of the present invention that successfully prevents or reduces the severity of symptoms associated with attention deficit disorder, with or without hyperactivity, when the patient also suffers from anxiety, depression, obesity, drug abuse/addiction, alcohol abuse, sleep disorders, TIC disorder, or behavioral/cognitive symptoms of Alzheimer's disease.

As used herein, the term “TIC disorder” refers to a one or more disorders, which include Tourette's Disorder, Chronic Motor or Vocal Tic Disorder, Transient Tic Disorder, and Tic Disorder Not Otherwise Specified. A more complete description of the symptoms and diagnostic criteria of TIC disorder is provided by DSM-IV (Diagnostic and Statistical Manual of Mental Disorders, 1994; 100-105), which is incorporated herein by reference.

It is contemplated that the therapeutically effective amount of a composition will depend on a number of factors, including by not limited to the age of the patient, immune status, race, and sex of the patient, and the severity of the condition/disease, and the past medical history of the patient, and always lies within the sound discretion of the administering physician. Generally, the total daily dose of the compounds of this invention administered to a patient in single or in divided doses can be in amounts, for example, 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day). Single dose compositions may contain such amounts or submultiples thereof to make up the daily dose. In general, treatment regimens according to the present invention comprise administration to a patient in need of such treatment 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day) in single or multiple doses. In addition, the total daily dose of the azapirone compounds, having no dopamine receptor activity (including ipsapirone and tandospirone), of this invention administered to a patient in need thereof, in single or in divided doses can be in amounts of 0.25-3.0 mg/kg of body weight/day.

Treatment regimens according to the present invention also include concurrently administering to a patient in need thereof mixtures, in single or divided doses, of two or more of the compounds of the present invention. When the compounds of the present invention are administered concurrently as mixtures, the therapeutically effective amount to be administered lies within the sound discretion of the administering physician; preferably, the compounds of the present invention may be administered to a patient in single or in divided doses in amounts of, for example, 0.25-0.75 mg/kg of body weight/day of gepirone (approximately 15 mg/day); 0.003-0.06 mg/kg of body weight/day of flesinoxan (approximately 0.4 mg/day), 0.5-3.0 mg/kg of body weight/day of adatanserin (approximately 120 mg/day), and 0.25-3.0 mg/kg of body weight/day of ipsapirone and tandospirone.

Alternatively, treatment regimens according to the present invention include sequentially administering to a patient in need thereof, in single or divided doses, two or more of the compounds of the present invention. An example of a sequential administration strategy includes administering a therapeutically effective amount of a first compound followed by, on the same day or a subsequent day, a single or divided dose of a therapeutically effective amount of one or more additional compounds. As used herein, the term “subsequent day” refers to any day ranging from the next day (>24 hours) to one week (≦168 hours) after administration of the previous compound. The term “same day” refers to any time frame ranging from immediately after administration of the previous compound to <24 hours after administration of the previous compound.

When the compounds of the present invention are administered sequentially as a part of a combination therapy, the therapeutically effective amount to be administered lies within the sound discretion of the administering physician; preferably, the compounds of the present invention may be administered to a patient in single or in divided doses in amounts of, for example, 0.25-0.75 mg/kg of body weight/day of gepirone (approximately 15 mg/day); 0.003-0.06 mg/kg of body weight/day of flesinoxan (approximately 0.4 mg/day), 0.5-3.0 mg/kg of body weight/day of adatanserin (approximately 120 mg/day), and 0.25-3.0 mg/kg of body weight/day of ipsapirone and tandospirone.

As used herein, the terms “treat”, “treating”, and “treatment” also embrace the terms alleviation and amelioration. In addition, it is also within the scope of the present invention to use the methods described and/or claimed herein for the prevention of attention deficit disorder, with or without hyperactivity, as well as the symptoms associated therewith. Moreover, the terms “treat”, “treating”, and “treatment” also may embrace prevention of attention deficit disorder.

Having generally described this invention, a further understanding can be obtained by reference to certain specific examples, which are provided herein for purposes of illustration only, and are not intended to be limiting unless otherwise specified.

### EXAMPLES

Attention Deficit/Hyperactivity Disorder can exist alone or comorbidity with other psychiatric disorders in children. In fact, ADHD is one of the most common comorbidities associated with other disorders. ADHD can exist in conjunction with TIC disorder, Anxiety disorders, and Depressive disorders.

### Example 1

### The Benefit of 5-HT_{1A} Partial Agonists in ADHD Alone

Experiments to gain FDA approval for this indication would involve two well-controlled, well-designed trials of the test drug in patients suffering with ADHD. A typical study would involve 50-100 children allocated 50% to the test drug and 50% to placebo. Medication would be given daily for approximately 8 weeks. Assessment of severity of ADHD symptoms would be completed prior to drug treatment and at regular intervals throughout the 8 weeks. The measurements and ratings would be similar to those mentioned above. Appropriate statistical procedures would be applied to the results. The study would be similar to that conducted by Greenhill et al (Pediatrics 2002;109:E39-52).

### Example 2

### The Benefit of 5-HT_{1A} Partial Agonists in Children with ADHD and TIC Disorder

The procedure here would be similar to Example 1. An experiment similar to that provided by Gadow et al (J Clin Psychopharm 2002;22:267-274) for methylphenidate (RITALIN™) could also be utilized.

### Example 3

### The Benefit of 5-HT_{1A} Partial Agonists in Children with ADHD and Anxiety Symptoms

The procedure here would be similar to Example 1. An experiment similar to those provided by Taylor et al (Psychol Med 1987;17:121-143) and/or Pliszka (J Am Acad Child Adolesc Psychiatry 1989;28:882-7) could also be utilized.

### Example 4

### The Benefit of 5-HT_{1A} Partial Agonist in Children with ADHD and Depressive Symptoms

The procedure here would be similar to Example 1. Additional diagnoses of depression would be made by DSM-IV criteria, and rating scales would include the Hamilton Depression Rating Scale (M. Hamilton, J Neurol Neurosurg Psychiatry 1960;23:56-62).

Numerous modifications and variations on the present invention are possible in light of the above teachings. It is therefore, to be understood that within the scope of the accompanying claims, the invention may be practiced otherwise than as specifically described herein.

What we claim is:

1. A method of treating attention deficit disorder, or symptoms thereof, in a patient in need thereof comprising administering a therapeutically effective amount of an azapirone, or a pharmaceutically acceptable salt thereof, wherein the azapirone has no dopamine receptor activity.

2. The method of claim 1, wherein the azapirone is selected from the group consisting of ipsapirone, tandospirone, and gepirone.

3. The method of claim 2, wherein the azapirone is ipsapirone.

4. The method of claim 3, wherein the therapeutically effective amount of ipsapirone is 0.25 to 3.0 mg per kg of body weight per day.

5. The method of claim 2, wherein the azapirone is tandospirone.

6. The method of claim 5, wherein the therapeutically effective amount of tandospirone is 0.25 to 3.0 mg per kg of body weight per day.

7. The method of claim 2, wherein the azapirone is gepirone.

8. The method of claim 7, wherein the therapeutically effective amount of gepirone is 0.25 to 0.75 mg per kg of body weight per day.

9. The method of claim 1, wherein the attention deficit disorder in the patient is further associated with hyperactivity.

10. The method of claim 1, wherein the azapirone is administered with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

11. The method of claim 1, wherein the azapirone is administered with methylphenidate.

12. The method of claim 1, wherein the azapirone is administered with a pharmaceutically acceptable carrier.

13. The method of claim 1, wherein said administering is selected from the group consisting of oral, rectal, nasal, parenteral, intracisternal, intravaginal, intraperitoneal, sublingual, topical, and bucal.

14. The method of claim 13, wherein said administering is oral or parenteral.

15. The method of claim 1, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

16. The method of claim 1, wherein the patient in need thereof also suffers from one or more disorders selected from the group consisting of anxiety, depression, and TIC disorder.

17. A method of treating attention deficit disorder, or symptoms thereof, in a patient in need thereof comprising administering a therapeutically effective amount of an adamantyl aryl piperazinyl carboxamide or heteroaryl piperazinyl carboxamide 5-HT_{1A} receptor agonist, or a pharmaceutically acceptable salt thereof.

18. The method of claim 17, wherein the attention deficit disorder in the patient is further associated with hyperactivity.

19. The method of claim 17, wherein the adamantyl aryl piperazinyl carboxamide or heteroaryl piperazinyl carboxamide is adatanserin.

20. The method of claim 17, wherein the adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

21. The method of claim 17, wherein the adamantyl aryl piperazinyl carboxamide or heteroaryl piperazinyl carboxamide is administered with methylphenidate.

22. The method of claim 17, wherein the adamantyl aryl piperazinyl carboxamide or heteroaryl piperazinyl carboxamide is administered with a pharmaceutically acceptable carrier.

23. The method of claim 17, wherein said administering is selected from the group consisting of oral, rectal, nasal, parenteral, intracisternal, intravaginal, intraperitoneal, sublingual, topical, and bucal.

24. The method of claim 23, wherein said administering is oral or parenteral.

25. The method of claim 17, wherein the therapeutically effective amount of the adamantyl aryl piperazinyl carboxamide or heteroaryl piperazinyl carboxamide is 0.003 to 0.06 mg per kg of body weight per day.

26. The method of claim 17, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

27. The method of claim 17, wherein the patient in need thereof also suffers from one or more disorders selected from the group consisting of anxiety, depression, and TIC disorder.

28. A method of treating attention deficit disorder, or symptoms thereof, in a patient in need thereof comprising administering a therapeutically effective amount of a hetrobicyclic-aryl-piperazine 5-HT_{1A} receptor agonist, or a pharmaceutically acceptable salt thereof.

29. The method of claim 28, wherein the hetrobicyclic-aryl-piperazine is flesinoxan.

30. The method of claim 28, wherein the attention deficit disorder in the patient is further associated with hyperactivity.

31. The method of claim 28, wherein the hetrobicyclic-aryl-piperazine is administered with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

32. The method of claim 28, wherein the hetrobicyclic-aryl-piperazine is administered with methylphenidate.

33. The method of claim 28, wherein the hetrobicyclic-aryl-piperazine is administered with a pharmaceutically acceptable carrier.

34. The method of claim 28, wherein said administering is selected from the group consisting of oral, rectal, nasal, parenteral, intracisternal, intravaginal, intraperitoneal, sublingual, topical, and bucal.

35. The method of claim 34, wherein said administering is oral or parenteral.

36. The method of claim 28, wherein the therapeutically effective amount of the hetrobicyclic-aryl-piperazine is 0.5 to 3.0 mg per kg of body weight per day.

37. The method of claim 28, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

38. The method of claim 28, wherein the patient in need thereof also suffers from one or more disorders selected from the group consisting of anxiety, depression, and TIC disorder.

39. A method of treating attention deficit disorder, or symptoms thereof, in a patient in need thereof comprising administering a therapeutically effective amount of two or more compounds selected from the group consisting of geprione, ipsapirone, tandospirone, flesinoxan, and adatanserin.

40. The method of claim 39, wherein the attention deficit disorder in the patient is further associated with hyperactivity.

41. The method of claim 39, wherein the compounds are administered with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

42. The method of claim 39, wherein the hetrobicyclic-aryl-piperazine is administered with methylphenidate.

43. The method of claim 39, wherein the compounds are administered with a pharmaceutically acceptable carrier.

44. The method of claim 39, wherein said administering is selected from the group consisting of oral, rectal, nasal, parenteral, intracisternal, intravaginal, intraperitoneal, sublingual, topical, and bucal.

45. The method of claim 44, wherein said administering is oral or parenteral.

46. The method of claim 39, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

47. The method of claim 39, wherein the patient in need thereof also suffers from one or more disorders selected from the group consisting of anxiety, depression, and TIC disorder.

48. The method of claim 39, wherein said two or more compounds are administered concurrently.

49. The method of claim 39, wherein said two or more compounds are administered sequentially.

50. The method of claim 49, wherein said two or more compounds are administered on the same day.

51. The method of claim 49, wherein said two or more compounds are administered on subsequent days.
